(19) United States
(12) Patent Application Publication
(10) Pub. No.: US 2013/0085557 A1
(43) Pub. Date: Apr. 4, 2013

(54) STIMULUS ELECTRODE FOR BIOLOGICAL TISSUE AND METHOD OF PRODUCING THE SAME

(71) Applicant: NIDEK CO., LTD., Gamagori-shi (JP)

(72) Inventor: Yasuo TERASAWA, Gamagori (JP)

(73) Assignee: NIDEK CO., LTD., Gamagori-shi (JP)

(21) Appl. No.: 13/630,317

(22) Filed: Sep. 28, 2012

(30) Foreign Application Priority Data
Sep. 30, 2011 (JP) 2011-215939

(51) Int. Cl. A61N 1/05; B23K 26/00

(52) U.S. Cl. 607/116; 219/121.85

(57) ABSTRACT

A method of producing a stimulus electrode for a biological tissue includes irradiating a surface of an electrode with an ultrashort pulse laser to form a plurality of deformations.

### CROSS-REFERENCE TO RELATED APPLICATIONS

This application claims priority to and the benefit of Japan Patent Application No. 2011-215939, filed on Sep. 30, 2011, in the Japan Patent Office, each of the disclosure, claims, abstract, and drawings of which is incorporated herein by reference in its entirety.

### BACKGROUND

1. Technical Field

The present disclosure relates to a stimulus electrode to deliver electrical stimulation to a region of a biological tissue and a method of producing the stimulus electrode.

2. Foundation

An electrical stimulation device for a living body that applies electrical stimulation to a region of a biological tissue so as to regulate a bodily function is being studied. Two typical examples of devices for a living body that deliver electrical stimulation are a cochlear implant and a cardiac pacemaker. The cochlear implant stimulates the auditory nerve of a patient so as to transmit sound vibration to auditory ossicles. The cardiac pacemaker is implanted in a patient's chest, and electrically stimulates a heart muscle, thus controlling an irregular heartbeat. Another typical electrical stimulation device for a living body is a vision restoration assist apparatus (as disclosed in JP-A-2009-082496). The vision restoration assist apparatus outputs an electrical stimulation pulse signal (electric charge) via an electrode, and electrically stimulates a cell forming a retina, thus promoting vision restoration.

The electrical stimulation device delivers a predetermined amount of electric charge via the electrode so as to sufficiently stimulate the cell. The stimulus electrode is arranged in a compact space in vivo. In view of this, it is preferred that the electrode have a minimized size to reduce the patient's discomfort. The ability of the stimulus electrode to deliver denser electric charges becomes higher in proportion to a surface area of the electrode.

Especially, the electrical stimulation device with a plurality of electrodes needs to have a reduced size of the electrode and an expanded surface area. Therefore, this application is directed toward increasing the surface area of the electrode by generating a redox reaction to make its surface rough by etching (as disclosed in JP-A-2011-030734).

This type of application ensures the increased surface area of the electrode. However, this method has the following problems: 1. This limits the kinds of electrode material that can be surface treated with this method. 2. The reproducibility of the surface conditioning pattern of the electrode may be difficult. The present disclosure provides a stimulus electrode for biological a tissue that is more stable in property and a method of producing the stimulus electrode.

### SUMMARY

According to a first aspect of the present disclosure, there is provided a method of producing a stimulus electrode for a biological tissue. The method includes irradiating a surface of an electrode with an ultrashort pulse laser to form a plurality of deformations. According to a second aspect of the present disclosure, there is provided a stimulus electrode for a biological tissue. The stimulus electrode includes a surface on which a plurality of deformations is formed by an irradiation of an ultrashort pulse laser.

### BRIEF DESCRIPTION OF THE DRAWINGS

FIG. 1A is a side view of an electrode;

FIG. 1B is a top view of the electrode;

FIG. 2A is a cross-sectional side view of the electrode before a surface treatment;

FIG. 2B is a cross-sectional side view of the electrode during the surface treatment;

FIG. 3 is a block diagram of a laser irradiation device;

FIG. 4A is a top view of an electrode according to a modification of the surface treatment on the electrode;

FIG. 4B is a top view of an electrode according to a modification of the surface treatment on the electrode;

FIG. 4C is a top view of an electrode according to a modification of the surface treatment on the electrode;

FIG. 4D is a top view of an electrode according to a modification of the surface treatment on the electrode;

FIG. 5A is an explanatory diagram of a procedure of producing an electrode substrate of a vision restoration assist apparatus;

FIG. 5B is an explanatory diagram of the procedure of producing the electrode substrate of the vision restoration assist apparatus;

FIG. 5C is an explanatory diagram of the procedure of producing the electrode substrate of the vision restoration assist apparatus;

FIG. 6 is a schematic block diagram of the vision restoration assist apparatus;

FIG. 7A is an observation of a surface condition of an electrode according to Example 1 (a magnification of 150 times);

FIG. 7B is an observation of the surface condition of the electrode according to Example 1 (a magnification of 500 times);

FIG. 8A is an observation of a surface condition of an electrode according to Example 2 (a magnification of 100 times); and

FIG. 8B is an observation of the surface condition of the electrode according to Example 2 (a magnification of 2000 times).

### DETAILED DESCRIPTION OF EMBODIMENTS

Preferred embodiments of the present invention will be described below with reference to the accompanying drawings, in which like reference characters designate similar or identical parts throughout the several views thereof.

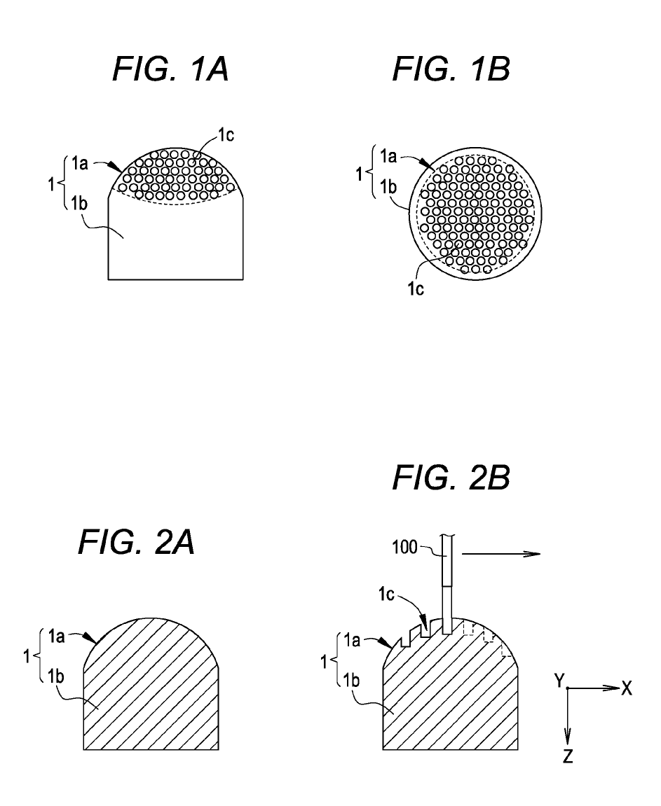

FIG. 1A is a side view of an electrode 1 according to this embodiment. FIG. 1B is a top view of the electrode 1 according to this embodiment.

The electrode 1 has a predetermined three-dimensional shape. The electrode 1 has a distal end (an upper end) 1a that has a curved surface with a predetermined curvature (a curvature radius). The distal end 1a of the electrode 1 includes a plurality of holes 1c. In contrast, the electrode 1 has a side face 1b without the holes 1c. Thus, the side face 1b is smoother than a surface at the distal end 1a.

While the holes 1c are not formed all over the surface of the electrode 1, the holes 1c are formed only on the surface of the electrode 1 at the distal end 1a. This increases only density of the electric charges that can be discharged from a local region at the distal end 1a. This configuration enables appropriate electrical stimulation to the local region of a biological tissue.

Next, a method of producing the electrode 1 will be described. FIG. 2A is a cross-sectional side view of the electrode 1 before a surface treatment on the electrode. FIG. 2B is a cross-sectional side view of the electrode 1 during the surface treatment on the electrode. First, an outline of the electrode 1 is formed. A metal bulk material with biocompatibility is cut in a predetermined length. Then, well-known metal processing (such as cutting work) adjusts a shape and a size of the cut material, thus providing a predetermined outline of the electrode 1. The following method may also be available. First a metal bulk material (a rod material) is processed into an electrode shape. Then, the metal bulk material is cut in order to provide the electrode 1 with a predetermined outline.

Here, the three-dimensional electrode 1 is formed to have a bullet shape that has an outer diameter of 100 to 500 μm and a height of 100 to 500 μm. The electrode 1 employs a well-known metal with biocompatibility as a forming material. Here, a platinum (Pt) material is chosen. The electrode 1 may be formed of other materials such as gold, titanium nitride, pure iridium, iridium oxide, tantalum, and a mixture of these materials. In some embodiments, the electrode 1 is an alloy.

The electrode 1 may be formed in various sizes and shapes according to its usage using well-known metal processing. For example, the metal processing includes a cutting work, machining, lathing, filing and the like. This forms, for example, a plate-shaped electrode with a predetermined thickness. In some embodiments, the electrode has a solid volume space less than 10 mm³. In some embodiments, the solid volume space is less than 1 mm³. Next, a surface treatment is performed on the electrode 1 so as to increase a surface area (charge injection capability) of the electrode 1. In this embodiment, the surface of the electrode 1 is irradiated with an ultrashort pulse laser to form the plurality of holes 1c on the surface of the electrode 1, thus increasing the surface area (charge injection capability). The ultrashort pulse laser is a laser with a pulse width from nanosecond to femtosecond. In some embodiments, the pulse width is measured at the full-width half-maximum. In some embodiments, the pulse width ranges from 1 fs to 999 ps. In some embodiments, the pulse width ranges from 20 fs to 900 fs or from 100 fs to 700 fs. In some embodiments, the ultrashort pulse has a pulse energy ranging from 0.01 μJ to 10 mJ. In some embodiments, the pulse energy ranges from 0.1 μJ to 5 mJ or from 0.9 μJ to 2 mJ. In some embodiments, the ultrashort laser has pulse repetition frequency ranging from 100 Hz to 100 Mhz. In some embodiments, the pulse repetition frequency ranges from 100 kHz to 80 MHz or from 200 kHz to 20 MHz. In some embodiments, the pulse repetition frequency ranges from 500 Hz to 5 kHz or from 1 kHz to 4 kHz. Such features are available from mode locked lasers such as solid state (Ti:sapphire and the like) or dye mode locked lasers. In some embodiments, the laser pulse has a maximum intensity wavelength ranging from 700 nm to 1,200 nm or from 780 nm to 1,100 nm. In some embodiments, e.g., the maximum of the bandwidth of the laser pulse is 780 to 800 nm or 1020 to 1060 nm.

The ultrashort pulse laser with a short pulse width is used for the surface treatment on the electrode 1. This forms more holes 1c on the surface of the electrode 1. This also restricts heat, which is generated by laser irradiation, from propagating near the holes 1c, thus accurately forming the holes 1c. Using a series of ultrashort laser pulses having one or more features above makes it possible to avoid the reflow material, i.e., roughness induced by the creation of high temperatures within or adjacent to the region near the holes 1c.

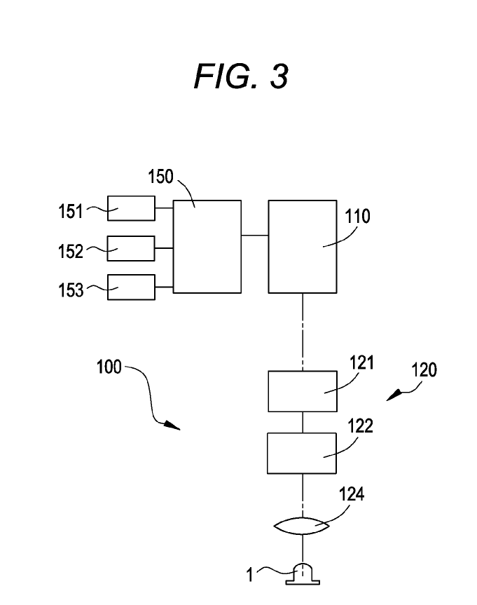

An exemplary laser irradiation device will be described. FIG. 3 is a block diagram of the laser irradiation device. The laser irradiation device 100 includes a laser light source 110, a laser irradiation optical system 120, and a controller 150. The laser light source 110 emits a pulse laser that causes breakdown at a focal point. The laser irradiation optical system 120 guides the laser and irradiates a target (the electrode 1) with the laser. The controller 150 drives the whole laser irradiation device 100.

The laser light source 110 employs a device that emits an ultrashort pulse laser with a pulse width from nanosecond to femtosecond. The ultrashort pulse laser generates plasma (causing breakdown) at the focal point of the laser (a laser absorption point). Emitting the laser cuts an object (a part of the electrode) at the focal point or waist of the pulse laser.

The irradiation optical system 120 includes a scanning unit (an optical scanner) 121, a focus shifting unit (a focus shifter) 122, and an objective lens 124. The scanning unit 121 two-dimensionally (in the XY direction) scans (deflects) the laser absorption point (a focus position) of the pulse laser on a target surface. The focus shifting unit 122 shifts the laser absorption point of the pulse laser in the optical axis direction (the Z direction). The objective lens 124 focuses the laser and forms an image on the target surface. The scanning unit 121 and the focus shifting unit 122 constitute a moving optical system that three-dimensionally moves the absorption point of the laser light toward the electrode 1.

The controller 150 controls operations of the whole laser irradiation device 100. The controller 150 is coupled to an input unit 151, a foot switch 152, a memory 153, and the like. The input unit 151 is used for setting a laser irradiation condition and a laser scanning condition. The foot switch 152 is used to input a trigger signal to begin laser irradiation. The memory 153 preliminarily stores various conditions.

The laser light source 110 irradiates the surface of the electrode 1 with a laser beam from above, thus causing breakdown at a laser absorption point of the laser. This causes mechanical destruction (for example, cracking) having a size of the absorption point on the surface of the electrode 1. As illustrated in FIG. 2B, the controller 150 controls the focus shifting unit 122 to shift the laser absorption point to the optical axis direction (the Z direction). Then, the hole 1c with a predetermined depth is formed on the distal end 1a of the electrode 1. The hole 1c is formed with at least one laser irradiation. The hole 1c is formed deeper by irradiating with the laser several times in different positions in the optical axis direction.

After forming the hole 1c in a predetermined position on the surface of the electrode 1, the scanning unit 121 and the focus shifting unit 122 are driven so as to move the laser irradiation position in a horizontal direction (the XY direction). Thus, another hole 1c is formed in a different position at the distal end 1a. Thus, three-dimensionally moving the laser absorption point allows the formation of a plurality of holes 1c at desired positions at the distal end 1a of the electrode 1 by laser irradiation.

In this embodiment, the controller 150 drives the moving optical system to adjust the laser irradiation position on the surface of the electrode 1, thus forming the plurality of holes 1c in different positions at the distal end 1a of the electrode 1. Another configuration is available for moving the laser irradiation position on the surface of the electrode 1. The configuration fixes the laser irradiation device 100 and moves the electrode 1 in a three dimensional direction. For example, the electrode 1 may be placed on a stage (not shown). Then, the controller 150 moves the stage in a three dimensional direction. As another configuration, the respective drive controls of the moving optical system 122 and the stage may be combined together. That is, any other configuration is available insofar as the laser irradiation position is adjusted relative to the electrode 1.

The hole 1c has a diameter, a depth, a positional relationship (the distance) with the adjacent hole 1c, which are determined with a irradiation condition. The irradiation condition may be preliminarily stored in the memory 153, and selected through the input unit 151. Alternatively, in the case where a condition to form the hole 1c is set through the input unit 151, the controller 150 may automatically determine another condition. For example, the controller 150 automatically sets a position where the adjacent holes 1c are formed depending on the set size of the holes 1c.

Further, in some embodiments, the laser irradiation position in the optical axis direction (the Z direction) be adjusted corresponding to the shape of the electrode 1. Inputting height information and the like of the electrode 1 to the memory 153 allows the controller 150 to recognize the shape of the electrode 1. For example, the height information of the electrode 1 is provided by obtaining variations (Z1−Z0=ΔZ) among heights of other positions (coordinate) (X1, Y1) at the distal end 1a relative to a height (Z0) of the center position (coordinate) (X0, Y0) at the distal end 1a of the electrode 1.

For example, as illustrated in FIGS. 1A to 1B and 2A to 2B, the electrode 1 with a three-dimensional shape has a height that varies from the periphery to the center. For this electrode 1, a focal length in the optical axis direction is adjusted corresponding to the shape (the height information) of the electrode 1. The information on the shape of the electrode 1 is preliminarily stored in the memory 153. In addition, the laser irradiation device 100 may include a detector (not shown), which detects the surface shape (height) of the electrode 1, and adjust the position of the laser irradiation device 100 or the electrode 1 in the optical axis direction based on a detection result of the detector.

A method that increases the surface area of the electrode using a conventional technique of a chemical reaction (etching) may cause variation in the surface condition of the electrode due to different kinds of electrode materials. The variation in the surface condition of the electrode 1 occurs even among electrodes with the same electrode material depending on their individual differences after the surface treatment.

In contrast, the present disclosure increases the surface area by irradiating the surface of the electrode 1 with the laser. Thus, the present disclosure sets a laser irradiation condition regardless of the kind of metallic material forming the electrode 1, thus ensuring an electrode with a constant surface condition. That is, the surface treatment using the ultrashort pulse laser ensures the electrodes 1 with the same surface condition regardless of individual differences of the electrodes 1 with high reproducibility.

In this embodiment, platinum is used as an electrode material. In some embodiments, the hole 1c in the electrode 1 has a diameter ranging from 5 μm to 30 μm, In some embodiments, the hole 1c in the electrode 1 has a diameter ranging from 5 μm to 100 μm, and a depth with an aspect ratio equal to or more than 1. The aspect ratio A is a value obtained as a ratio of a depth “a” of the hole 1c to the diameter “d” of the hole 1c, which is expressed by A=d/a. In some embodiments, the aspect ratio ranges from 0.02 to 2 or from 0.04 to 1.5. In some embodiments, the aspect ratio is the average value of the aspect ratio for the set of holes on the electrode.

A diameter smaller than 5 μm of the hole 1c makes it difficult to form the hole 1c on the surface of the electrode 1. This increases the possibility of variation (occurrence of local bias in an amount of increase in surface area) in the surface condition of the electrode 1. On the other hand, a diameter larger than 30 μm of the hole 1c reduces the number of the holes 1c to be formed on the electrode surface, thus tending to restrict the surface area of the electrode 1 from increasing.

The aspect ratio A smaller than 1 makes the depth of the hole 1c shallower, thus restricting the surface area of the electrode 1 from increasing. A larger aspect ratio makes the surface area of the electrode 1 increase more. However, this increases a delay time between an output electric charge from the surface (the upper portion 1b) of the electrode 1 and an output electric charge from inside the hole 1c. Preventing a large delay time is, in some embodiments, used so as not to reduce accuracy of the electric charge (an electrical pulse signal), which is output from the electrode 1. The diameter and the depth, which are described above, of the hole 1c are selected such that the diameter and the depth satisfy these characteristics. The diameter and the depth are combined with the shape and the material of the electrode as needed.

As described above, forming the plurality of holes 1c at the distal end 1a of the electrode 1 by laser irradiation increases the surface area of the electrode 1 (the distal end 1a), thus improving charge injection capability. This ensures the electrodes 1 with the same surface condition regardless of the kind of the electrode material or individual differences of the electrodes with high reproducibility. The holes 1c are formed in a region with the distal end 1a of the electrode 1 at the center. This concentrates injection charges on the distal end 1a of the electrode 1, thus applying accurate (pinpoint) electrical stimulation to the local region of the biological tissue. In contrast, no holes 1c is formed on the side face 1b of the electrode 1 without the laser irradiation. This increases the surface area of the electrode 1 at the distal end 1a without changing the outline shape of the side face of the electrode 1.

In FIGS. 1A and 1B, the holes 1c as examples, are formed such that distances (center-to-center distances) between the plurality of holes 1c, which are mutually adjacent, are constant. In this case, the surface area of the distal end 1a evenly increases, thus facilitating uniform current (electric charges) injection from the electrode 1. For example, a forming pattern of the hole 1c at the distal end 1a employs a square grid pattern, a houndstooth pattern, or the like. In some embodiments, the formed pattern is symmetrical, i.e., is has symmetry rather than being random. This makes the center-to-center distances between the adjacent holes 1c constant, and makes the center-to-center distances as short as possible. In this case, the increased number of holes 1c are formed at the distal end la, thus facilitating the increased surface areas. The center-to-center distances between the plurality of adjacent holes 1c might not be constant. Any center-to-center distances may be set corresponding to a laser irradiation pattern.

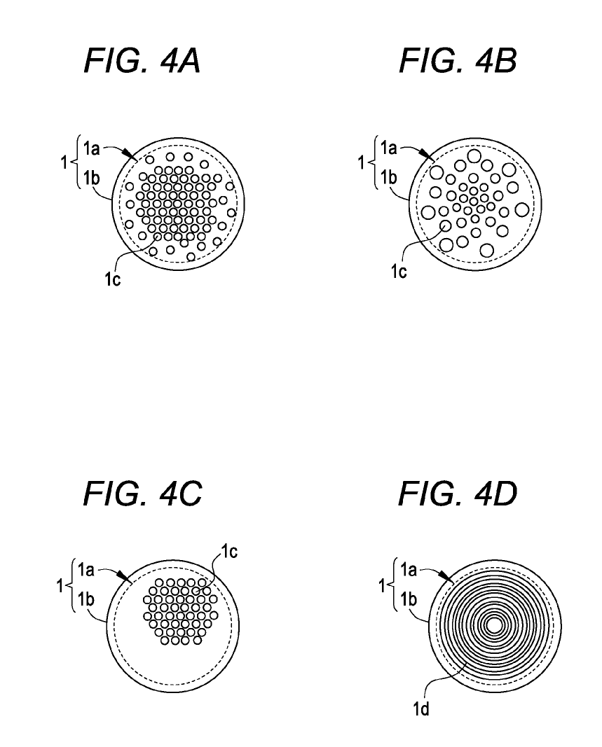

FIGS. 4A to 4D illustrate modifications of the surface condition of the electrode 1 on which the laser irradiation device 100 has performed a surface treatment. Here, top views (diagrams seen from the distal end 1a side) of the electrodes 1 are illustrated. For example, the holes 1c may be formed as follows. As illustrated in FIG. 4A, the holes 1c are densely formed near the center of the distal end 1a but are sparsely formed toward the outside by determination of the laser irradiation pattern. The density of the holes 1c near the center of the distal end 1a of the electrode 1 is increased compared with the density of the holes 1c at the periphery. This increases density of the distributed charges in the center of the distal end 1a where the holes 1c are densely formed when electrical stimulation is performed. Thus, stimulation to a local region is, in some embodiments, performed.

The diameters of the holes 1c may be formed to vary from the center of the distal end 1a to the periphery. For example, as illustrated in FIG. 4B, the diameter of the hole 1c may become larger from the center of the distal end 1a to the periphery. While the depth of the hole 1c formed near the center of the distal end 1a may be set to deeper, the depth of the hole 1c may become shallower from the center to the periphery of the electrode 1.

Furthermore, as illustrated in FIG. 4C, the hole 1c may be formed in any local region on the electrode surface. This increases charge injection capability in a region including the local region of the electrode 1 with increased surface area as the center. As illustrated in FIG. 4D, the surface area may be increased. A laser irradiation is continuously performed on regions from the center of the distal end 1a toward the periphery so as to form a spiral-shaped groove 1d.

In addition, controlling machining (laser processing) corresponding to a shape, usage, and the like of the electrode 1 forms recesses with various shapes on the surface of the electrode 1 using the holes or the grooves. This arbitrarily increases the surface area of the electrode 1, thus arbitrarily increasing charge injection capability of the electrode 1. A known processing to improve charge injection capability may be further performed after the above surface treatment forms the holes 1c on the surface of the electrode 1. For example, processes such as platinum black plating or forming an iridium oxide film may be performed.

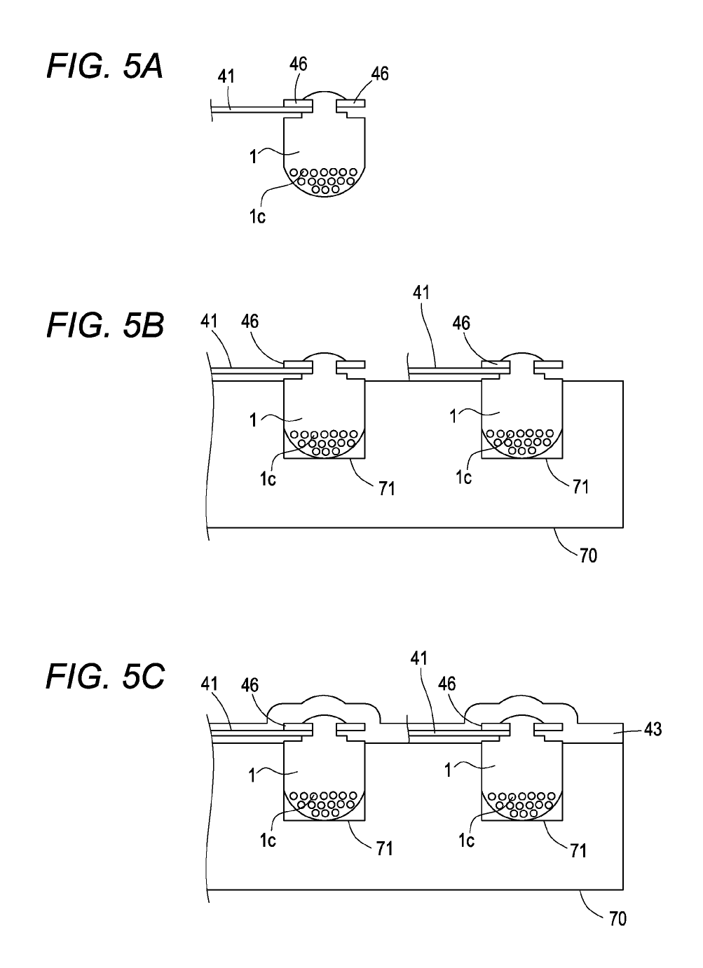

Next, an exemplary electrical stimulation device including the electrode 1 where the above surface treatment has been performed will be described. Here, an exemplary vision restoration assist apparatus, which applies electrical stimulation to a patient's retina, will be described. FIGS. 5A to 5C are explanatory views of a procedure of producing an electrode substrate that is used in the vision restoration assist apparatus.

As illustrated in FIG. 5A, a base of the electrode 1 (a reference numeral is omitted) includes the plurality of holes 1c formed on its surface. Distal ends of a plurality of wires 41 with insulating coating are placed on the base. Then, a welding technique couples (welds) them together. The welding technique includes laser welding, resistance welding, and the like. At this time, coatings of insulating resin in the welded portions of the wires 41 are removed by heat of welding. This electrically couples the electrode 1 to the wires 41. Coupling the electrode 1 and the wires 41 may be mechanical coupling using pressure bonding or the like.

Next, the electrode 1 is inserted into a well-known washer 46 from the base of the electrode 1 until the washer 46 is brought into contact with the wires 41. Then, the base is pressed so as to sandwich (couple) the wires 41 between the electrode 1 and the washer 46. The processing is individually performed for the respective electrodes 1, thus electrically coupling the wires 41 to the respective electrodes 1.

Next, as illustrated in FIG. 5B, a substrate 43, which includes the electrode 1, is formed using a tool 70. The tool 70 includes a plurality of holes 71 formed by laser processing or machining The hole 71 has an inner diameter larger than an outer diameter of the electrode 1. The hole 71 has a diameter that matches the diameter of each of the electrodes 1. The holes 71 have the depth that is the same as or slightly shallower than the height of the respective electrodes 1. This places the wires 41 such that the wires 41 are not brought into contact with the tool 70 when the electrode 1 is mounted or placed on the tool 70. Alternatively, the wires 41 are disposed on the tool 70.

The substrate 43 is formed of resin after the electrodes 1 are arranged in the holes 71 of the above tool 70. For example, as illustrated in FIG. 5C, the tool 70 on which the electrodes 1 are placed is disposed inside a deposition device (not shown). Next, a substrate material (for example, Parylene® with biocompatibility and a high insulation property is grown on the tool 70. This forms a resin layer with a predetermined thickness corresponding to the shape of the tool 70. The resin layer is formed in a plate shape. This forms the flexible substrate 43 that covers the wires 41.

At this time, in this embodiment, the surface treatment by laser on the side face lb of the electrode 1 is not performed, thus preserving the outline shape of the electrode 1. Consequently, gaps are not formed between the electrode 1 and the hole 71, thus preventing the resin from flowing into between the electrode 1 and the hole 71. In view of this, an operation (process) for removing resin adhered to the electrode 1 is omitted. Accordingly, the substrate 43 is more efficiently produced.

In the above description, the substrate 43 includes the electrode 1 on which the surface treatment has preliminarily been performed. In addition, the surface treatment may be performed on the electrode 1 by laser irradiation after the electrode 1 is implanted in the substrate 43. This increases the surface area (charge injection capability) of the electrode 1 (the distal end 1a). This process arranges the respective electrode 1 in a predetermined position on the substrate 43 such that the distal end 1a where the plurality of holes 1c is formed projects from the surface of the substrate 43.

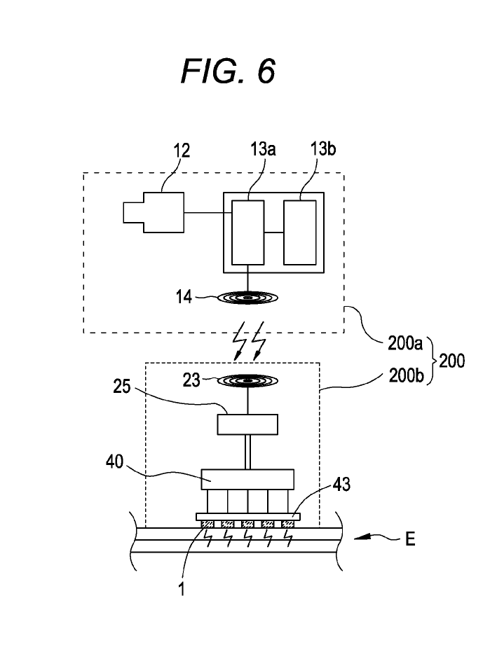

Next, a configuration of the vision restoration assist apparatus will be described. FIG. 6 is a schematic block diagram of the vision restoration assist apparatus. The vision restoration assist apparatus 200 includes an external device 200a, which is mounted outside of a patient's body, and an implantable device 200b, which is implanted inside the patient's body and delivers electrical stimulation to biological tissues. The external device 200a includes a photographing device 12, which photographs the external world, a converting unit 13a for a pulse signal, a power source 13b, which supplies electric power to the whole vision restoration assist apparatus, and a transmitting unit 14, which transmits data for electrical stimulation pulse to the implantable device 200b side. The converting unit 13a for a pulse signal converts photograph data (image data) from the photographing device 12 to a signal (data for electrical stimulation pulse) in a predetermined frequency band that is needed for the patient to see the image.

The implantable device 200b includes a receiving unit 23, which receives a signal from the external device 200a, a controller 25, an electronic circuit 40, and the electrodes 1, which are embedded in the substrate 43. The controller 25 generates an electrical stimulation pulse to be delivered to the respective electrodes 1 based on data for the electrical stimulation pulse, which is received by the receiving unit 23. The controller 25 controls operation of the whole implantable device 200b. The electronic circuit 40 delivers the electrical stimulation pulse to the respective electrodes 1 based on a signal from the controller 25. The electronic circuit 40 is electrically coupled to the respective electrodes 1 via the wire 41.

Photograph data (image data) of an object, which is photographed by the photographing device 12, is transmitted to the converting unit 13a for the pulse signal. The converting unit 13a for the pulse signal converts the photograph data of the object to data for the electrical stimulation pulse. Then, the converting unit 13a for the pulse signal superimposes the data for the electrical stimulation pulse on a carrier wave supplied from the power source 13b, thus transmitting an electromagnetic wave to the implantable device 200b through the transmitting unit 14.

The implantable device 200b receives the signal transmitted from the external device 200a at the receiving unit 23, and transmits the signal to the controller 25. The controller 25 generates electrical stimulation pulses, which are delivered to the respective electrodes 1, and various control signals based on the data for the electrical stimulation pulse, which is received by the receiving unit 23, and transmits the generated signals and pulses to the electronic circuit 40.

The electronic circuit 40 outputs an electrical stimulation pulse from each electrode 1 based on a received signal. The electrical stimulation pulse output from each electrode 1 stimulates a cell forming a retina E (see FIG. 6). This method provides vision for the patient. At this time, in this embodiment, the surface area of the distal end 1a of the electrode 1 is increased by the surface treatment by the laser irradiation, and the electrode 1 with higher charge injection capability is used. Accordingly, this increases charge density at the distal end 1a of the electrode 1, thus performing accurate electrical stimulation to a local region of the retina.

The electrode 1 on which the aforementioned surface treatment is performed by laser irradiation is a typical electrical stimulation device for biological tissues. For example, the electrode 1 is used in an electrical stimulation device such as a pacemaker and a cochlear implant. This improves charge injection capability without increasing the size of the electrode 1. The improved charge injection capability of the electrode 1 has an advantage in decreasing the size of the electrode 1. The downsized electrode 1 reduces the discomfort of the patient in which the electrode 1 is to be implanted. This also allows arranging more electrodes 1 in a limited area of biological tissues, thus improving accuracy of the electrical stimulation to the biological tissues.

In the above description, the irradiation of the ultrashort pulse laser generates the mechanical destruction. The mechanical destruction forms the deformation (recess or groove) on the electrode 1, thus increasing the surface area. In addition, irradiating the surface of the electrode 1 with the femtosecond laser may form the recess (groove) with a periodic structure, thus increasing the surface area. The periodic structure is formed such that incident light and reflected light of the laser mutually interfere in the case where an object (electrode) is irradiated with an intense femtosecond laser that is close to a threshold value of the processing. Although the pulse energies noted above are useable, in some embodiments, the pulse energy ranges from 0.5 to 5.0 μJ or from 1.0 to 2.5 μJ. The periodic structure is constituted of a plurality of grooves. The groove has a space frequency and a depth at the same level as a wavelength of the laser, thus increasing the surface area of the electrode. For example, platinum allows many periodic structures with both a pitch (distance) and a depth of several hundred nm to be formed on the surface (electrode surface) corresponding to a laser wavelength. The pitch and the depth of the periodic structure are at the same level as the laser wavelength. In some embodiments, the laser wavelength is altered by frequency up-conversion or down-conversion, using, e.g., a nonlinear optical crystal.

The pitch and the depth of the periodic structure, which are determined by the laser wavelength, equal to or more than 1 nm increases the surface area of the electrode. In some embodiments, pitch and depth of the periodic structure are formed from 200 nm to 1200 nm. This appropriately increases the surface area of the electrode (metal). The periodic structure formed by laser irradiation is formed regardless of the shape of the electrode. Especially, the periodic structure appropriately ensures an increased surface area of the thin electrode 1 where forming the hole or the groove by mechanical destruction is difficult. With another electrode shape (for example, a bullet shape), densely forming the periodic structure in a region that is desired to have high charge density appropriately ensures improved charge injection capability. Furthermore, combination of: a surface treatment where a plurality of holes or grooves are formed by mechanical destruction; and a surface treatment where the periodic structure is formed by femtosecond laser irradiation may be used to increase the surface area (charge injection capability) of the electrode 1.

Next, experimental results where the ultrashort pulse laser is actually used for the surface treatment of the electrode 1 will be described.

### EXAMPLE 1

A bullet-shaped electrode that was formed of platinum (Pt) as a material and had a diameter of about 500 μm and a height of about 300 μm was used as an electrode. The laser irradiation device (LWL-3030-T10) that was used was made by SIGMA KOKI CO., LTD. The laser irradiation condition included an oscillation frequency of 200 KHz, an average output equal to or more than 400 mW, a pulse width equal to or less than 500 fs, and a pulse repetition frequency of 200 kHz. The laser beam was collected by an objective lens with magnification of 20 times. The electrode is irradiated with a femtosecond laser with a beam diameter of 15 μm at its top surface. The electrode surface condition was observed through Scanning Electron Microscope (SEM). The laser irradiation position (a distance and a location of a hole) with respect to the surface of the electrode was set using CAD.

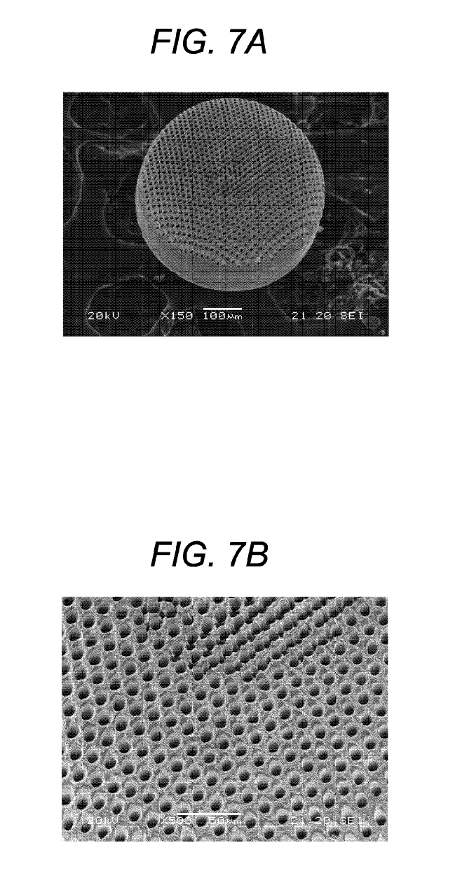

FIGS. 7A and 7B illustrate SEM observation results of the electrode surface after the surface treatment of the electrode 1. FIG. 7A is a photograph image of the electrode surface with magnification of 150 times. FIG. 7B is a photograph image of the electrode surface with magnification of 500 times. FIG. 7A shows that a plurality of holes with uniform shape is formed on the electrode at its top surface. This demonstrates that the above surface treatment by laser irradiation improved charge injection capability of the electrode by about two to three times.

### EXAMPLE 2

A platinum foil (plate electrode) with a thickness of 20 μm was used as an electrode. The laser irradiation device that was used was the same as that in Example 1. The irradiation condition of the femtosecond laser included an average output of 1.2 μJ/pulse, a processing speed of 2 mm/s, a spiral pitch of 10 μm, and with trepanning. The laser beam was collected by an objective lens with magnification of 5 times. The same SEM as that in Example 1 was used for the observation of the electrode surface.

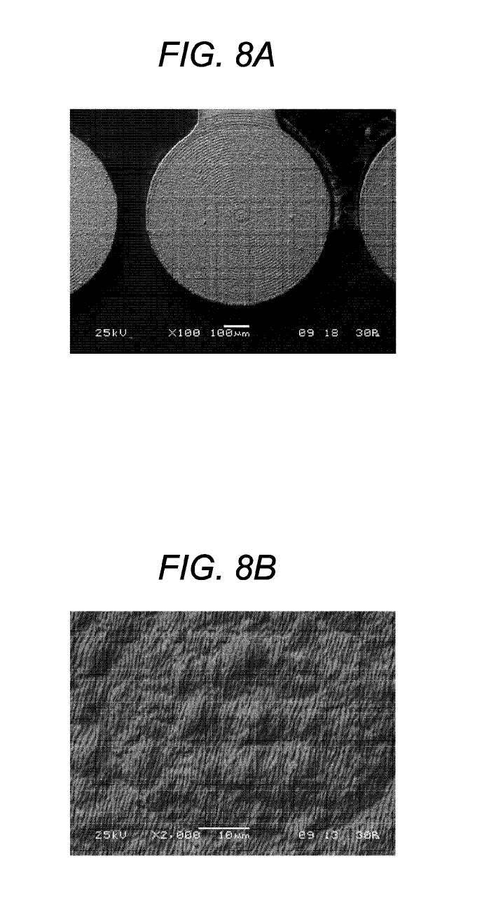

FIGS. 8A and 8B show appearance evaluations as observation results of the SEM. FIG. 8A is a photograph image with magnification of 100 times. FIG. 8B is a photograph image of the electrode surface with magnification of 2000 times. From FIGS. 8A and 8B, the periodic structure in the irradiation area of the femtosecond laser was confirmed. The periodic structure formed the groove on the electrode surface by concave-convex surface with complicated shapes. By this increased electrode area, improvement of charge injection capability is expected

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. A method of producing a stimulus electrode for a biological tissue, comprising
irradiating a surface of the electrode with an ultrashort pulse laser to form a plurality of deformations.

2. The method according to claim 1, wherein
the irradiating irradiates the surface of the electrode with the laser such that a density of the deformations formed in a center portion of the surface of the electrode is denser than a density of the deformations formed in a peripheral portion of the surface of the electrode.

3. The method according to claim 2, wherein
the irradiating forms the plurality of deformations in a shape of a hole and/or a groove on the surface of the electrode.

4. The method according to claim 3, wherein
the irradiating forms the electrode with a three-dimensional shape, the plurality of holes formed on the surface of the electrode having a diameter equal to or more than 5 μm and equal to or less than 30 μm, and an aspect ratio being equal to or more than 1.

5. The method according to claim 4, wherein
the irradiating irradiates the surface of the electrode with the laser such that the plurality of holes is arranged in a square grid pattern or a houndstooth pattern on the surface of the electrode.

6. The method according to claim 5, wherein
the electrode performs an electrical stimulation of a cell, the cell constituting a retina in a patient so as to promote a vision restoration,
the method further comprising
arranging a plurality of the electrodes on a substrate, the substrate being embedded inside a living body.

7. The method according to claim 1, further comprising
the irradiating irradiates the surface of the electrode with the laser from above the electrode.

8. A method of producing a stimulus electrode for a biological tissue, comprising
controlling an output of an ultrashort pulse laser by a laser unit using a control unit such that respective adjacent deformations are arranged to have a constant distance, a plurality of the deformations being formed on a surface of the electrode.

9. The method according to claim 8, wherein
the electrode has a three-dimensional shape, and
the control unit controls a position of the irradiation of a laser light in a depth direction, the electrode being irradiated with the laser light corresponding to a height of the electrode.

10. The method according to claim 9, wherein
the laser unit includes a femtosecond laser device, the femtosecond laser device outputting a femtosecond laser pulses.

11. A stimulus electrode for a biological tissue, comprising
a surface on which a plurality of deformations are formed by an irradiation of an ultrashort pulse laser.

12. The stimulus electrode according to claim 11, wherein
a density of the deformations formed in the center portion of the surface of the electrode is higher than a density of the deformations formed at a peripheral portion of the surface of the electrode.

13. The stimulus electrode according to claim 12, wherein
the plurality of deformations includes one of a plurality of recesses and a plurality of grooves.

14. The stimulus electrode according to claim 13, wherein
the electrode has a three-dimensional shape,
the plurality of holes formed on the surface of the electrode has a diameter equal to or more than 5 μm and equal to or less than 30 μm, and
an aspect ratio is equal to or more than 1.

15. The stimulus electrode according to claim 11, wherein
the plurality of deformations has a periodic structure, the periodic structure being formed by the ultrashort pulse laser.

16. The stimulus electrode according to claim 15, wherein
the periodic structure has a space frequency and a depth of equal to or more than 200 nm and equal to or less than 1200 nm.

17. A method of producing a stimulus electrode for a biological tissue, comprising
irradiating a surface of the electrode with ultrashort laser pulses from an ultrashort pulse laser to form a plurality of deformations, wherein the electrode has a solids volume less than 1 mm³, wherein the ultrashort pulse laser has a pulse power ranging from 0.01 μJ to 10 mJ and a pulse width at full width half maximum ranging from 1 fs to 999 ps.

18. The method of claim 1, wherein the deformations, in the plurality of deformations, have a dimension ranging from 5 μm to 100 μm.

19. The method of claim 1, wherein the deformations, in the plurality of deformation, have an aspect ratio ranging from 0.02 to 2.

20. The method of claim 1, wherein the ultrashort pulse laser has a pulse power ranging from 0.9 μJ to 2 mJ and a pulse width at full width half maximum ranging from 100 fs to 700 fs.
